(19) United States
(12) Patent Application Publication
(10) Pub. No.: US 2017/0095582 A1
(43) Pub. Date: Apr. 6, 2017

(54) Integrated Flip Chip Device Array

(71) Applicant: Sensor Electronic Technology, Inc., Columbia, SC (US)

(72) Inventors: Michael Shur, Latham, NY (US); Maxim S. Shatalov, Columbia, SC (US); Alexander Dobrinsky, Loudonville, NY (US); Emmanuel Lakios, Elgin, SC (US)

(73) Assignee: Sensor Electronic Technology, Inc., Columbia, SC (US)

(21) Appl. No.: 15/283,490

(22) Filed: Oct. 3, 2016

Related U.S. Application Data
(60) Provisional application No. 62/236,051, filed on Oct. 1, 2015.

(51) Int. Cl. A61L 2/08; H01L 27/15; A61L 2/10; H01L 27/12

(52) U.S. Cl. CPC A61L 2/084; H01L 27/1214; H01L 27/156; A61L 2/10; A61L 2202/11

(57) ABSTRACT

An optoelectronic device module with improved light emission of approximately 4π steradians is provided. In one embodiment, the optoelectronic device module includes a first and a second set of optoelectronic devices. Each optoelectronic device includes a first contact and a second contact. A contact element including a first lateral side and a second lateral side connects the optoelectronic devices. The first contact of each optoelectronic device in the first set of optoelectronic devices is connected to the first lateral side of the contact element and the first contact of each optoelectronic device in the second set of optoelectronic devices is connected to the second lateral side of the contact element.

### REFERENCE TO RELATED APPLICATIONS

The current application claims the benefit of U.S. Provisional Application No. 62/236,051, which was filed on 1 Oct. 2015, and which is hereby incorporated by reference.

### TECHNICAL FIELD

The disclosure relates generally to optoelectronic devices, and more particularly, to an optoelectronic device module with an array of optoelectronic devices for improved light emission.

### BACKGROUND ART

A light emitting diode (LED) is a semiconductor device that includes an N-type semiconductor and a P-type semiconductor, and emits light through recombination of holes and electrons. Such an LED has been used in a wide range of applications such as display devices, traffic lights, and backlight units. Further, considering the potential merits of lower power consumption and longer lifespan than existing electric bulbs or fluorescent lamps, the application range of LEDs has been expanded to general lighting by replacing existing incandescent lamps and fluorescent lamps.

The LED may be used in an LED module. The LED module is manufactured through a process of fabricating an LED chip at a wafer level, a packaging process, and a modulation process. Specifically, semiconductor layers are grown on a substrate such as a sapphire substrate, and subjected to a wafer-level patterning process to fabricate LED chips having electrode pads, followed by division into individual chips (chip fabrication process). After mounting the individual chips on a lead frame or a printed circuit board, the electrode pads are electrically connected to lead terminals via bonding wires, and the LED chips are covered by a molding member, thereby providing an LED package (packaging process). The LED package is mounted on a circuit board such as a metal core printed circuit board (MC-PCB), thereby providing an LED module such as a light source module (modulation process).

A typical LED mounted on a PCB radiates light in a portion of a sphere surrounding the LED module (which constitutes a solid angle of about 2π steradians).

### SUMMARY OF THE INVENTION

Embodiments of the present invention provide an optoelectronic device module with the light emission into 4π steradians. In addition, aspects of the present invention incorporate various embodiments for controlling and operating an optoelectronic device module.

Aspects of the invention are directed towards an optoelectronic device module with improved light emission of approximately 4π steradians. In one embodiment, the optoelectronic device module includes a first and a second set of optoelectronic devices. Each optoelectronic device includes a first contact and a second contact. A contact element including a first lateral side and a second lateral side connects the optoelectronic devices. The first contact of each optoelectronic device in the first set of optoelectronic devices is connected to the first lateral side of the contact element and the first contact of each optoelectronic device in the second set of optoelectronic devices is connected to the second lateral side of the contact element.

A first aspect of the invention provides an optoelectronic device module, comprising: a first and a second set of optoelectronic devices, wherein each optoelectronic device includes a first contact and a second contact; and a contact element including a first lateral side and a second lateral side, wherein the first contact of each optoelectronic device in the first set of optoelectronic devices is connected to the first lateral side of the contact element and the first contact of each optoelectronic device in the second set of optoelectronic devices is connected to the second lateral side of the contact element.

A second aspect of the invention provides a LED module, comprising: a first and a second set of LEDs, wherein each LED includes a first contact and a second contact; and a contact element including a first lateral side and a second lateral side, wherein the first contact of each LED in the first set of LEDs is connected to the first lateral side of the contact element and the first contact of each LEDs in the second set of LEDs is connected to the second lateral side of the contact element, wherein a light emitted by the first and second set of LEDs is at least 4π steradians.

A third aspect of the invention provides a disinfection module, comprising: a container including an inlet for receiving a fluid and an outlet for releasing the fluid contained within the container; a set of sensors configured to determine a transparency of the fluid within the container; and a set of optoelectronic device modules for emitting radiation to disinfect the fluid within the container, each of the optoelectronic device modules comprising: a first and a second set of optoelectronic devices, wherein each optoelectronic device includes a first contact and a second contact; and a contact element including a first lateral side and a second lateral side, wherein the first contact of each optoelectronic device in the first set of optoelectronic devices is connected to the first lateral side of the contact element and the first contact of each optoelectronic device in the second set of optoelectronic devices is connected to the second lateral side of the contact element.

The illustrative aspects of the invention are designed to solve one or more of the problems herein described and/or one or more other problems not discussed.

### BRIEF DESCRIPTION OF THE DRAWINGS

These and other features of the disclosure will be more readily understood from the following detailed description of the various aspects of the invention taken in conjunction with the accompanying drawings that depict various aspects of the invention.

FIG. 1 shows an illustrative optoelectronic device module according to an embodiment.

FIGS. 2A and 2B show electrical diagrams of illustrative optoelectronic device modules according to embodiments.

FIG. 3 shows a three-dimensional perspective view of an illustrative optoelectronic device module according to an embodiment.

FIG. 4 shows a three-dimensional perspective view of an illustrative optoelectronic device module according to an embodiment.

FIG. 5 shows a three-dimensional perspective view of an illustrative optoelectronic device module according to an embodiment.

FIG. 6A shows an illustrative printed circuit board (PCB) including a set of optoelectronic devices according to an embodiment, while FIG. 6B shows an illustrative optoelectronic device according to an embodiment, and FIG. 6C shows an illustrative PCB including a set of optoelectronic devices according to an embodiment.

FIG. 7A shows an illustrative substrate wafer including a set of optoelectronic devices according to an embodiment, while FIG. 7B shows an illustrative PCB including the substrate wafer according to an embodiment, and FIG. 7C shows an illustrative PCB including substrate wafers on both sides according to an embodiment.

FIG. 8 shows a circuit diagram of an illustrative PCB according to an embodiment.

FIG. 9A shows a top view of an illustrative PCB according to an embodiment, and FIG. 9B shows a circuit diagram of an illustrative contact within an opening of the PCB according to an embodiment.

FIG. 10A shows an illustrative optoelectronic device according to an embodiment, and FIG. 10B shows an illustrative PCB according to an embodiment.

FIGS. 11A and 11B show top and side perspective views of an illustrative PCB according to embodiments.

FIG. 12 shows an illustrative optoelectronic module according to an embodiment.

FIG. 13 shows an illustrative optoelectronic module according to an embodiment.

FIG. 14 shows an illustrative disinfection module including a set of optoelectronic modules according to an embodiment.

FIG. 15 shows an illustrative flow diagram for fabricating a circuit that comprises an optoelectronic module according to one the various embodiments described herein.

It is noted that the drawings may not be to scale. The drawings are intended to depict only typical aspects of the invention, and therefore should not be considered as limiting the scope of the invention. In the drawings, like numbering represents like elements between the drawings.

### DETAILED DESCRIPTION OF THE INVENTION

As indicated above, embodiments of the present invention provide an optoelectronic device module with the light emission into 4π steradians. In addition, aspects of the present invention incorporate various embodiments for controlling and operating an optoelectronic device module.

The optoelectronic device modules of the various embodiments described herein are suitable for use with a variety of optoelectronic devices. Examples of optoelectronic devices include, but are not limited to, light emitting devices, light emitting diodes (LEDs), including conventional and super luminescent LEDs, ultraviolet LEDs, light emitting solid state lasers, laser diodes, photodetectors, photodiodes, diodes, including bipolar diodes and unipolar diodes, transistors, including bipolar transistors, unipolar transistors, and high-electron mobility transistors (HEMTs), and/or the like. These examples of optoelectronic devices can be configured to emit electromagnetic radiation from a light generating structure such as an active region upon application of a bias. The electromagnetic radiation emitted by these optoelectronic devices can comprise a peak wavelength within any range of wavelengths, including visible light, ultraviolet radiation, deep ultraviolet radiation, infrared light, and/or the like. For example, these optoelectronic devices can emit radiation having a dominant wavelength within the ultraviolet range of wavelengths. As an illustration, the dominant wavelength can be within a range of wavelengths of approximately 210 nanometers (nm) to approximately 350 nm. In an embodiment, the optoelectronic device can be configured to be a sensor.

As used herein, unless otherwise noted, the term “set” means one or more (i.e., at least one) and the phrase “any solution” means any now known or later developed solution. It is understood that, unless otherwise specified, each value is approximate and each range of values included herein is inclusive of the end values defining the range. As used herein, unless otherwise noted, the term “approximately” is inclusive of values within +/− ten percent of the stated value, while the term “substantially” is inclusive of values within +/− five percent of the stated value. Unless otherwise stated, two values are “similar” when the smaller value is within +/− twenty-five percent of the larger value. A value, y, is on the order of a stated value, x, when the value y satisfies the formula 0.1x≦y≦10x.

As also used herein, a layer is a transparent layer when the layer allows at least ten percent of radiation having a target wavelength, which is radiated at a normal incidence to an interface of the layer, to pass there through. Furthermore, as used herein, a layer is a reflective layer when the layer reflects at least ten percent of radiation having a target wavelength, which is radiated at a normal incidence to an interface of the layer. In an embodiment, the target wavelength of the radiation corresponds to a wavelength of radiation emitted or sensed (e.g., peak wavelength +/− five nanometers) by an active region of an optoelectronic device during operation of the device. For a given layer, the wavelength can be measured in a material of consideration and can depend on a refractive index of the material. Additionally, as used herein, a contact is considered “ohmic” when the contact exhibits close to linear current-voltage behavior over a relevant range of currents/voltages to enable use of a linear dependence to approximate the current-voltage relation through the contact region within the relevant range of currents/voltages to a desired accuracy (e.g., +/− one percent).

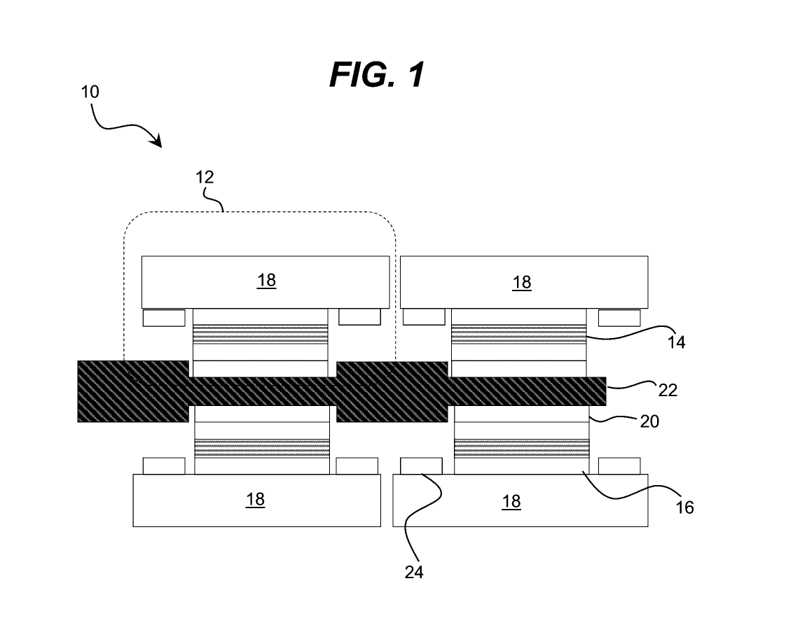

Turning to the drawings, FIG. 1 shows an illustrative optoelectronic device module 10 including a set of optoelectronic devices 12 according to an embodiment. Although four optoelectronic devices 12 are shown, it is understood that the optoelectronic device module 10 can include any number of optoelectronic devices 12. In an embodiment, at least one of the optoelectronic devices 12 is an LED. For example, at least one of the optoelectronic devices 12 can be an UV LED. In a more particular illustrative embodiment, each optoelectronic device 12 can be a group III-V materials based device, in which some or all of the various layers are formed of elements selected from the group III-V materials system. In a still more particular illustrative embodiment, the various layers of the optoelectronic device 12 are formed of group III nitride based materials. Group III nitride materials comprise one or more group III elements (e.g., boron (B), aluminum (Al), gallium (Ga), and indium (In)) and nitrogen (N), such that B_{W}Al_{X}Ga_{Y}In_{Z}N, where 0≦W, X, Y, Z≦1, and W+X+Y+Z=1. Illustrative group III nitride materials include binary, ternary and quaternary alloys such as, AlN, GaN, InN, BN, AlGaN, AlInN, AlBN, AlGaInN, AlGaBN, AlInBN, and AlGaInBN with any molar fraction of group III elements.

Each optoelectronic device 12 includes an active region 14 (e.g., a series of alternating quantum wells and barriers). The active region 14 can be composed of In_{y}AlₓGa_{1-x-y}N, Ga_{z}In_{y}AlₓB_{1-x-y-x}N, an AlₓGa₁₋ₓN semiconductor alloy, or the like. Similarly, a semiconductor layer 16 can be composed of an In_{y}AlₓGa_{1-x-y}N alloy, a Ga_{z}In_{y}AlₓB_{1-x-y-z}N alloy, or the like. The molar fractions given by x, y, and z can vary between the various layers 14, 16. When the optoelectronic device 12 is configured to be operated in a flip chip configuration, such as shown in FIG. 1, the substrate 18 can be transparent to the target electromagnetic radiation. Although not shown, a buffer layer can be located between the substrate 18 and the semiconductor layer 16, and the buffer layer also can be transparent to the target electromagnetic radiation. To this extent, an embodiment of the substrate 18 is formed of sapphire. However, it is understood that the substrate 18 can be formed of any suitable material including, for example, silicon carbide (SiC), silicon (Si), bulk GaN, bulk AlN, bulk or a film of AlGaN, bulk or a film of BN, AlON, LiGaO₂, LiAlO₂, aluminum oxinitride (AlOₓN_{y}), MgAl₂O₄, GaAs, Ge, or another suitable material. Furthermore, a surface of the substrate 12 can be substantially flat or patterned using any solution.

The optoelectronic device 12 can further include a contact 20, which can form an ohmic contact to a contact element 22. The contact element 22 can be formed of any suitable reflective material including, for example aluminum, rhodium, a combination of metallic layers having aluminum, layers comprising metallic alloys having at least some metals being aluminum, and/or the like. In an embodiment, the contact 20 can include a p-type contact and the optoelectronic device 12 can include a set of n-type electrodes 24 to the n-type semiconductor layer 16. In another embodiment, the contact 20 can include an n-type contact and the optoelectronic device 12 can then include a set of p-type electrodes 24 to the p-type semiconductor layer 16. In an embodiment, although it is not shown, the contact 20 can comprise several conductive and reflective metal layers, while the electrodes 24 each comprise highly conductive metals. Since the optoelectronic module 10 includes several optoelectronic devices 12, the module 10 can radiate light at a wide angle. For example, the optoelectronic module 10 can radiate light at approximately 4π steradians. It is understood that each of the optoelectronic devices 12 can operate at the same or different wavelengths. Furthermore, each of the optoelectronic devices 12 can operate at different intensities, power, duration and/or the like. In an embodiment, the contact element 22 physically and/or electronically connects all of the optoelectronic devices 12 by connecting all of the contacts 20 of each device 12.

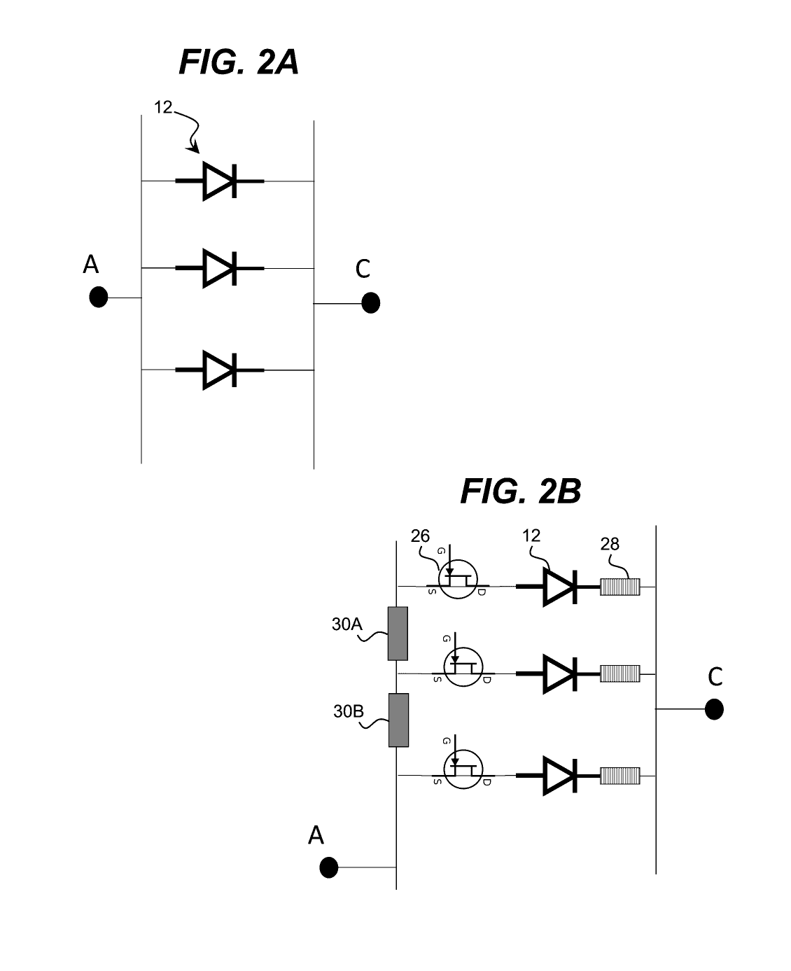

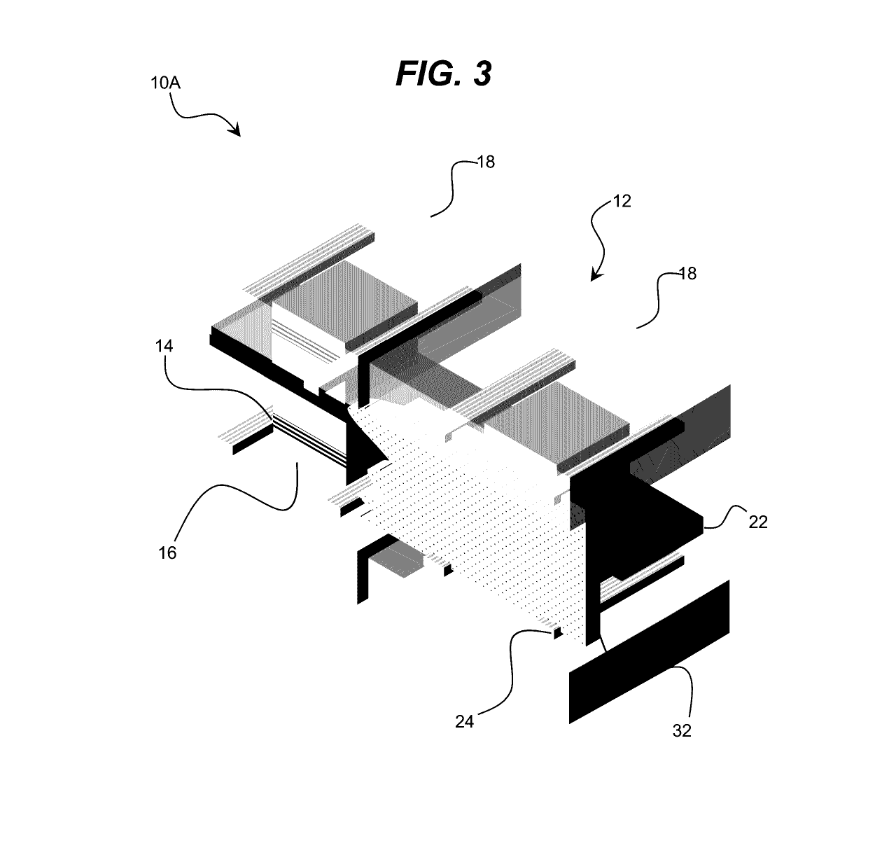

FIG. 2A shows an illustrative electrical diagram of a set of connected optoelectronic devices 12. For example, the optoelectronic devices 12 shown in FIG. 2 are diodes that are connected in parallel with all p-type contact sides of the device connected to the contact element 22 (FIG. 1). That is, the anode side of each device 12 (A) is the p-type contact side. Although the set of optoelectronic devices 12 are shown in parallel, it is understood that the electrical connections between the devices 12 can be serial connections or a combination of parallel and serial connections. Regardless, each of the devices 12 can include appropriate current control elements that allow for controlling each device individually and/or as a group. For example, in FIG. 2B, an illustrative electrical diagram of a set of optoelectronic devices 12 including a plurality of current control elements are shown. Each optoelectronic device 12 can include a transistor 26 and a resistor 28 that are connected in series with the optoelectronic device 12. Furthermore, a set of resistive elements (e.g., a resistor) 30A, 30B can be located on the anode side A of the optoelectronic devices 12.

Turning now to FIG. 3, a 3-dimensional (3D) perspective view of an illustrative optoelectronic device module 10A. The optoelectronic device module 10A includes all the features of the module 10 shown in FIG. 1. However, the module 10A shown in FIG. 3 also includes a contact 32 that connects all of the electrodes 24 of each of the devices 12. The contact 32 extends across the entire length of the module 10A. It is understood that only a portion of the contact 32 is shown in order to show the layers of each device 12. The contact 32 is of the same type as the electrodes 24. For example, if the electrodes 24 are n-type, then the contact 32 is also n-type. Furthermore, if the electrodes 24 are p-type, then the contact 32 is also p-type. The contact 32 can be used as a convenient way of connecting all the electrodes 24 to form a circuit.

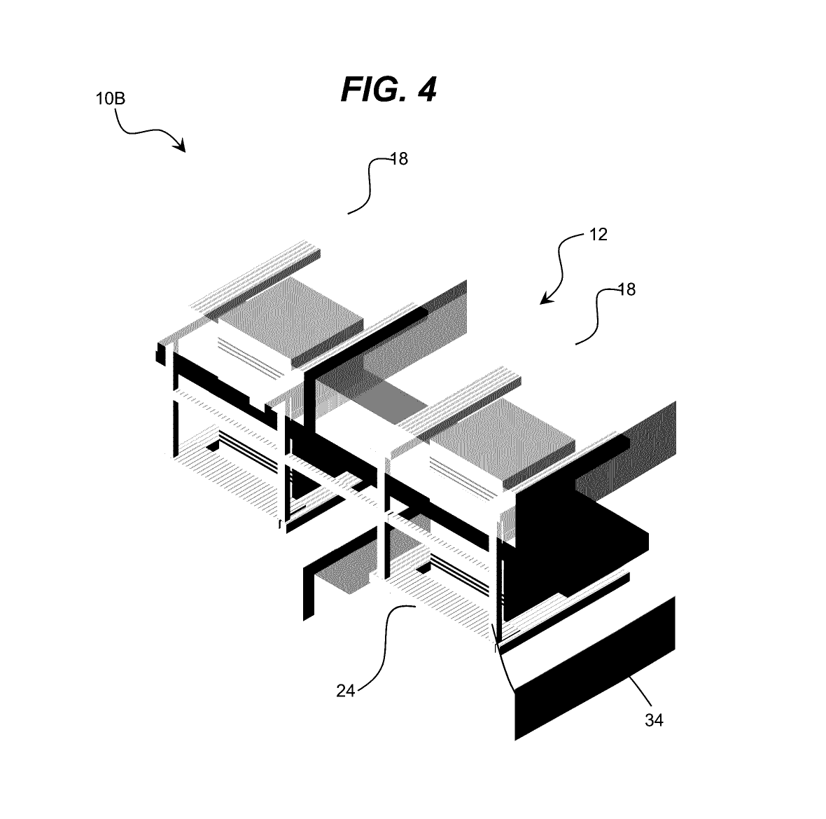

FIG. 4 shows another 3D perspective view of an illustrative optoelectronic device module 10B. In this embodiment, the electrodes 24 from each device 12 are connected by a mesh of electrodes 34, rather than the contact 32 shown in FIG. 3, to connect all the electrodes 24 to form a circuit. The use of a contact 32 (FIG. 3) or a mesh of electrodes 34 (FIG. 4) to connect all the electrodes 24 depends on the design specifications of the device, such as thermal management, the weight of the device, and/or the like.

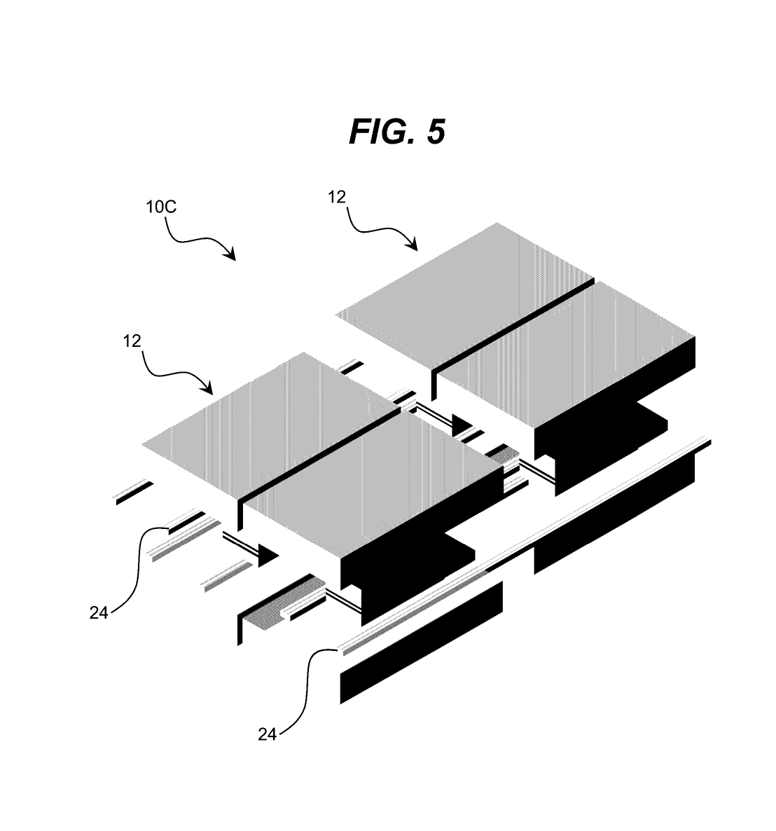

FIG. 5 shows yet another 3D perspective view of an illustrative optoelectronic device module 10C. However, in this embodiment, the electrodes 24 are continuous across each of the devices 12. In this embodiment, the devices are positioned differently in the physical space to allow for a specific emission pattern. As seen in FIG. 5, the electrodes 24 being cathodes, for example, can be connected together across the devices.

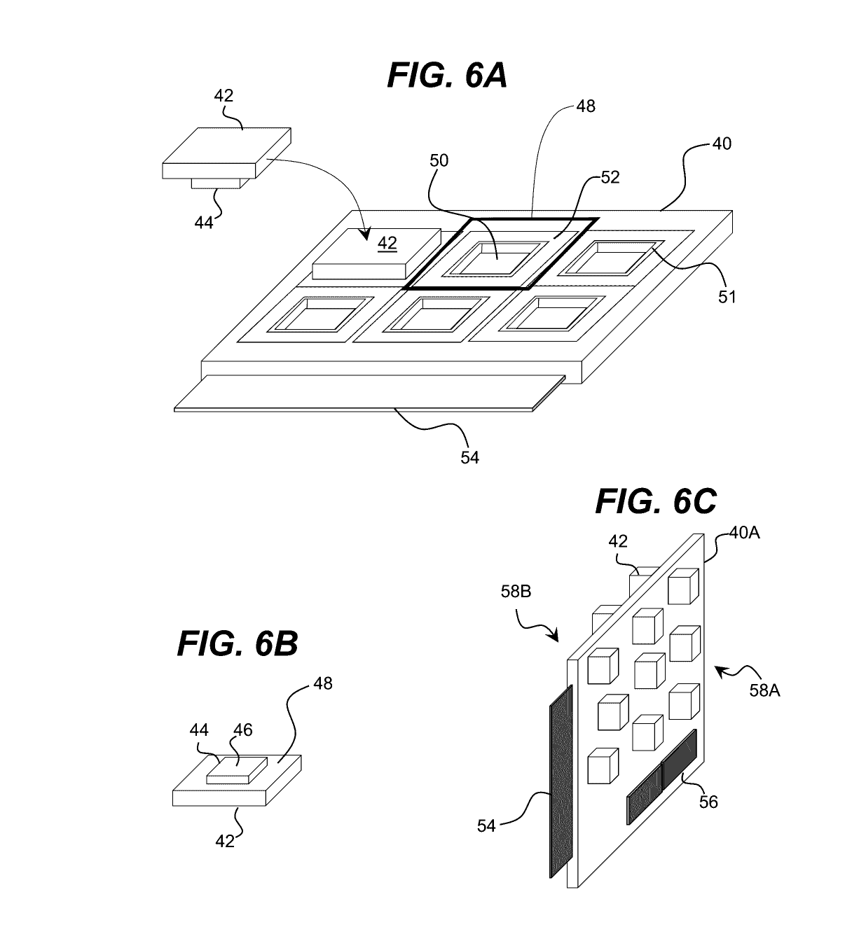

In another embodiment, a set of optoelectronic devices can be connected by a printed circuit board (PCB). For example, turning now to FIG. 6A, an illustrative PCB 40 is shown according to an embodiment. Each optoelectronic device 42 can include a mesa region 44 that extends from the device 42. FIG. 6B shows an illustrative mesa region 44 according to an embodiment. The mesa region 44 can include a p-type electrode 46, while a n-type electrode 48 surrounds the mesa region 44. Turning back to FIG. 6A, the PCB 40 can include a plurality of openings 48 that are configured for insertion of the mesa regions 44 of the set of optoelectronic devices 42. Each opening 48 includes a p-type contact 50 and a n-type contact 52 surrounds each opening 48, so that when the mesa region 44 of the optoelectronic device 42 is placed within the opening 48 (e.g., with the use of soldering), the p-type electrode 46 is connected to the p-type contact 50 and the n-type electrode 48 is connected to the n-type contact 52.

The p-type contact 50 and the n-type contact 52 are separated by a section 51 of the PCB 40 that is formed of an insulating material, such as FR-4 glass epoxy, and/or the like. It is understood that the remaining portions of the PCB 40 can be formed of this same material or another insulating material. The p-type contact 50 is within the PCB 40 in order to connect with a power supply 54. FIG. 6C shows an illustrative PCB 40 according to an embodiment. In this embodiment, the optoelectronic devices 42 can be connected to both sides of the PCB 40A. It is understood that both sides of the PCB 40A include the features of PCB 40 shown in FIG. 6A. In this embodiment, the PCB 40A can include openings 48 on a first lateral side 58A and a second lateral side 58B and each opening 48 is configured to receive an optoelectronic device 42. In an embodiment, as shown in FIG. 6C, the n-type contact 52 can be directly connected to a domain 56, which is capable of supplying power. Placement of optoelectronic devices on both sides 58A, 58B of the PCB 40A can result in a radiation pattern that spans approximately 4π steradians.

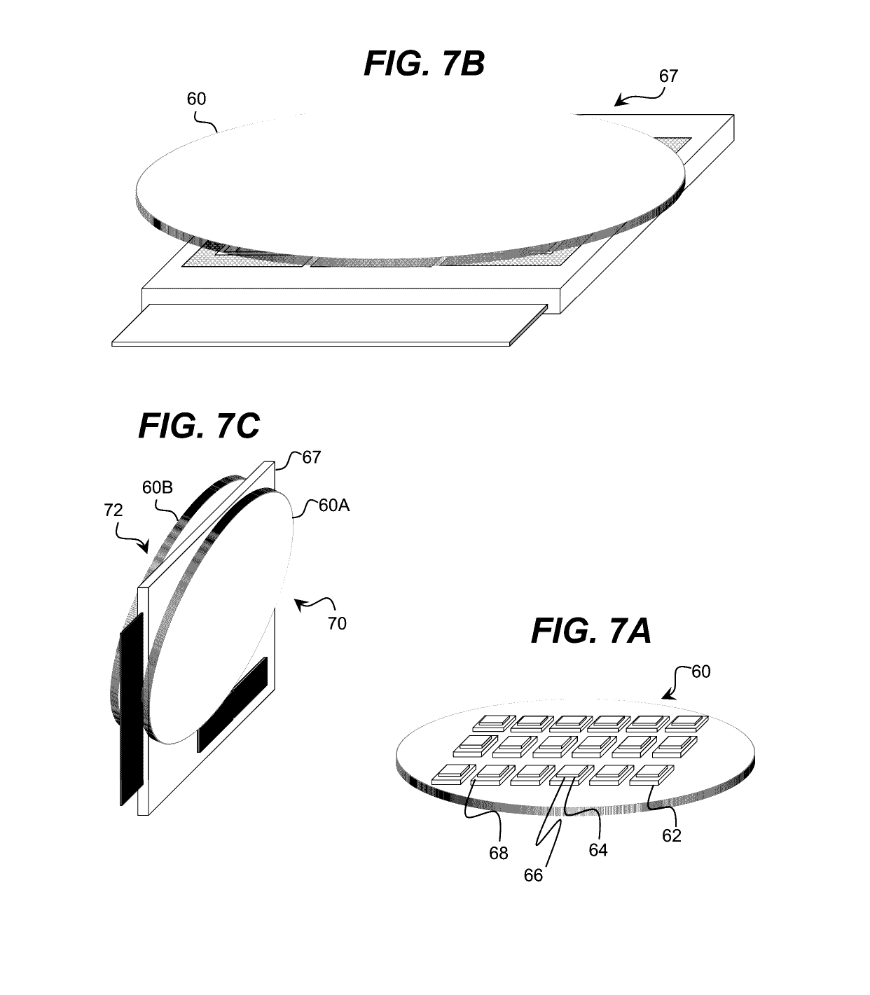

In another embodiment, the set of optoelectronic devices can be processed on a substrate wafer after the epitaxial growth and then attached to a PCB. For example, FIG. 7A shows an illustrative substrate wafer 60 according to an embodiment. The substrate wafer 60 includes a set of optoelectronic devices 62. Each optoelectronic device 62 is similar to the optoelectronic devices 42 shown in FIGS. 6A-6C and include a mesa region 64 with a p-type electrode 66 and a n-type electrode 68 surrounding the mesa region 64. Turning now to FIG. 7B, an illustrative PCB 67 including the substrate wafer 60 according to an embodiment is shown. Without separating the set of optoelectronic devices 62, the substrate wafer 60 including the set of optoelectronic devices 62 can be attached to the PCB 67. It is understood that the entire substrate wafer 60 or a subsection of the substrate wafer 60 can be attached to the PCB 67. Also, it is understood that the PCB 67 shown in FIG. 7B is similar to the PCB 40 shown in FIG. 6A.

By including the substrate wafer 60, the set of optoelectronic devices 62 are not physically separated from each other prior to attachment to the PCB 67. This allows for simpler processing of the optoelectronic devices 62. The PCB 67 could also have substrate wafers 60 attached to both sides. For example, in FIG. 7C, a first substrate wafer 60A is attached to a first lateral side 70 of the PCB 67 and a second substrate wafer 60B is attached to the second lateral side 72 of the PCB 67. It is understood that for a particularly designed PCB, the spacing between the optoelectronic devices 62 on the substrate wafer 60, the size of the mesa region for each optoelectronic device 62, and other physical characteristics can be selected to provide a match between the substrate wafer 60 and the PCB 67.

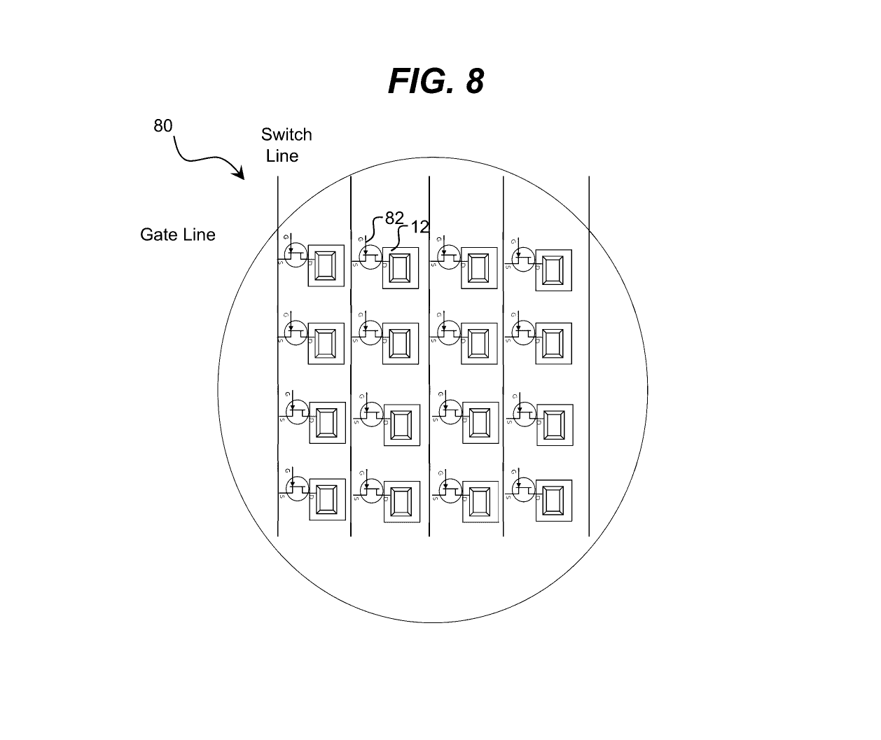

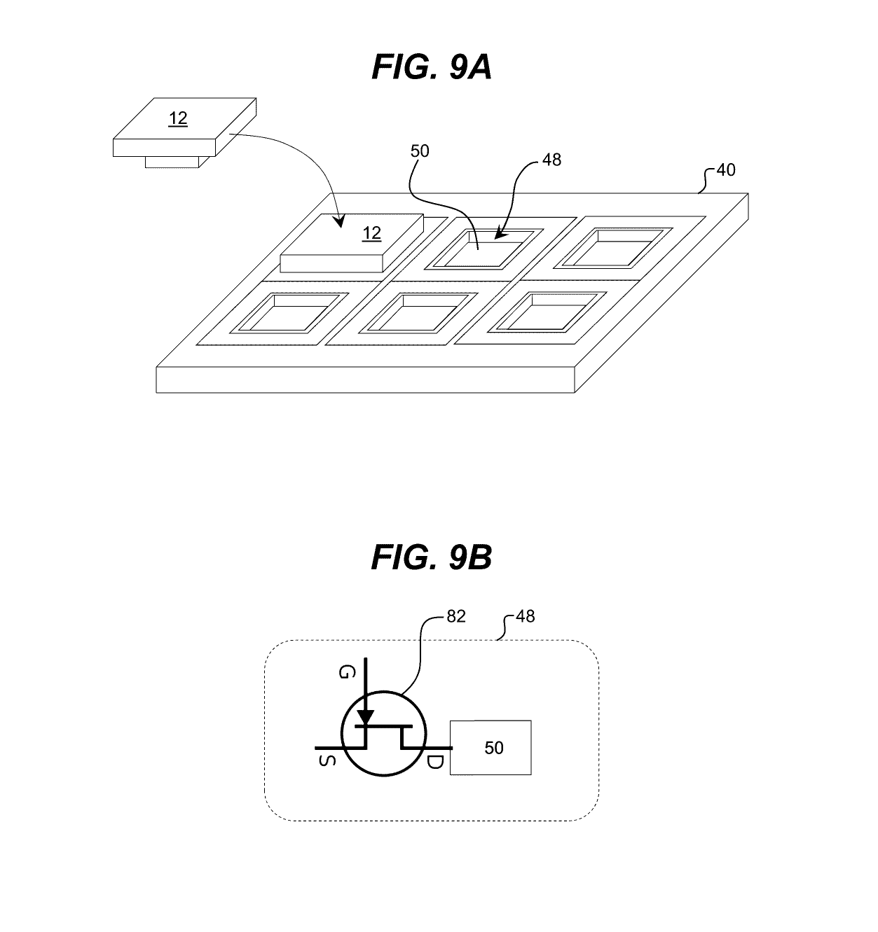

In another embodiment, an optoelectronic module can include a PCB that forms a thin film transistor (TFT)-like active matrix controller for controlling each optoelectronic device. It is understood that in this embodiment, the set of optoelectronic devices can be attached to the PCB using any approach. For example, FIG. 8 shows an illustrative optoelectronic module 80 according to an embodiment. Each optoelectronic device 12 can represent a pixel in a TFT active matrix network. Each optoelectronic device 12 includes a transistor element 82 that is attached to the device 12 in order to control the on/off switch characteristics of the optoelectronic device 12. The “Switch Line” and the “Gate Line” are used to control the devices 12. Similar to the operation of a TFT active matrix display, the set of optoelectronic devices 12 can be operated in a time dependent manner so that the entire module 80 has a characteristic refresh rate. As shown in FIGS. 9A and 9B, the transistor 82 can be connected to the p-type contact 50 within an opening 48 of the PCB 40.

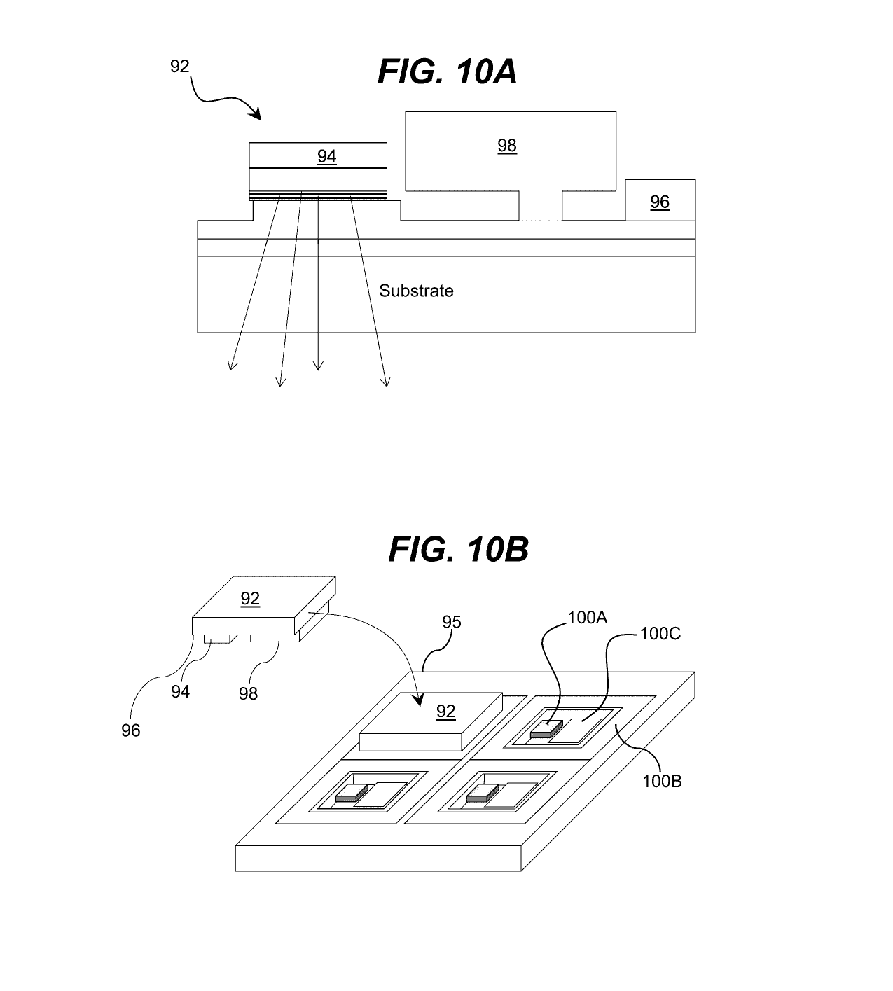

In another embodiment, an optoelectronic device can include semiconductor layers with a section that has high electron mobility transistor that allows for an on/off operation of the device. For example, as shown in FIG. 10A, an illustrative optoelectronic device 92 according to an embodiment is shown. In this embodiment, a drain contact 94 is connected to a source 96 through a gate 98 that can change the current characteristics of the device 92. For this device 92, as shown in FIG. 10B, a corresponding PCB 95 includes a set of contacts 100A, 100B, 100C for contacting the drain 94, the source 96 and the gate 98, respectively, for each device 92.

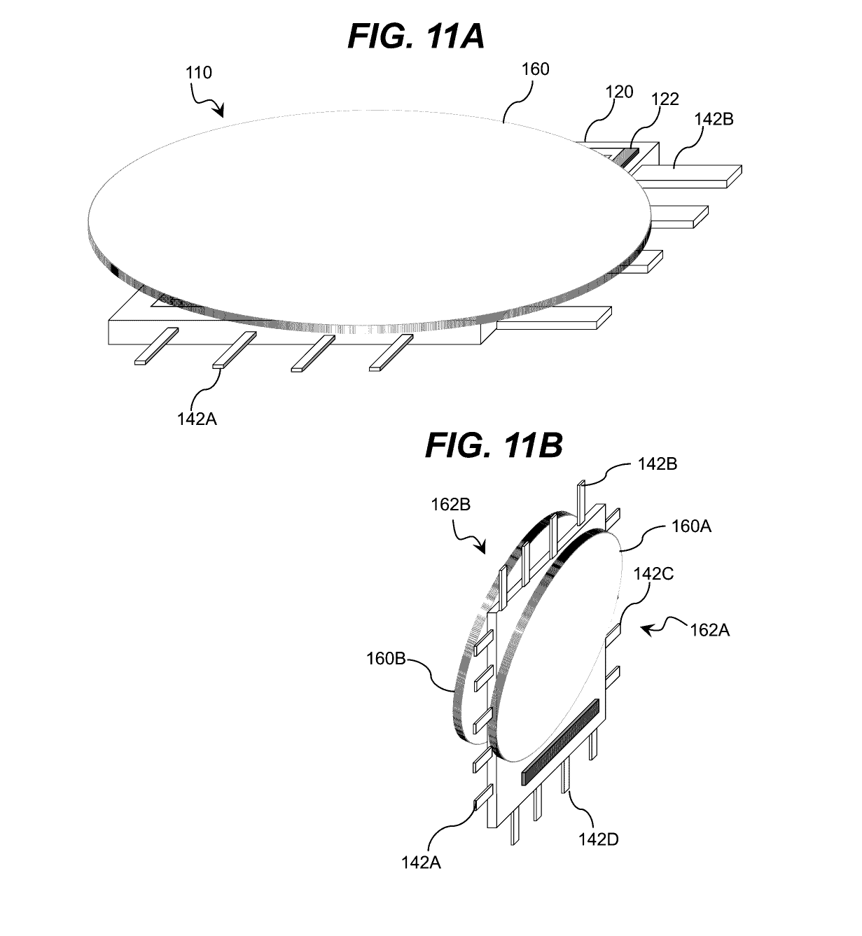

It is understood that the optoelectronic device 92 shown in FIG. 10A can be formed on a substrate wafer. For example, in FIG. 11A, an illustrative optoelectronic module 110 including a substrate wafer 160 is shown. The substrate wafer 160 can include a set of optoelectronic devices (not shown) that are connected to a PCB 120 with contacts that are organized in a TFT-like active matrix circuit with gate and switch drain lines 142A, 142B for controlling each device. The PCB 120 can also include a source/ground contact 122. In an embodiment, the contacts form equipotential surfaces where at least one of equipotential surface is ground.

It is understood that any devices that have similar contact characteristics can be combined together. For example, LED devices can be combined with sensing devices. In another embodiment, LED devices with different wavelength, emission pattern, or power can be combined together as long as each LED power is controlled through the active matrix. It is understood that a substrate wafer 160 can be connected to either side of the PCB 120. For example, turning now to FIG. 11B, it is understood that a first substrate wafer 160A is connected to a first lateral side 162A of the PCB 120, while a second substrate wafer 160B is connected to a second lateral 162B side of the PCB 120. Each wafer 160A, 160B can have individual gate and switch lines that extend from the PCB 120.

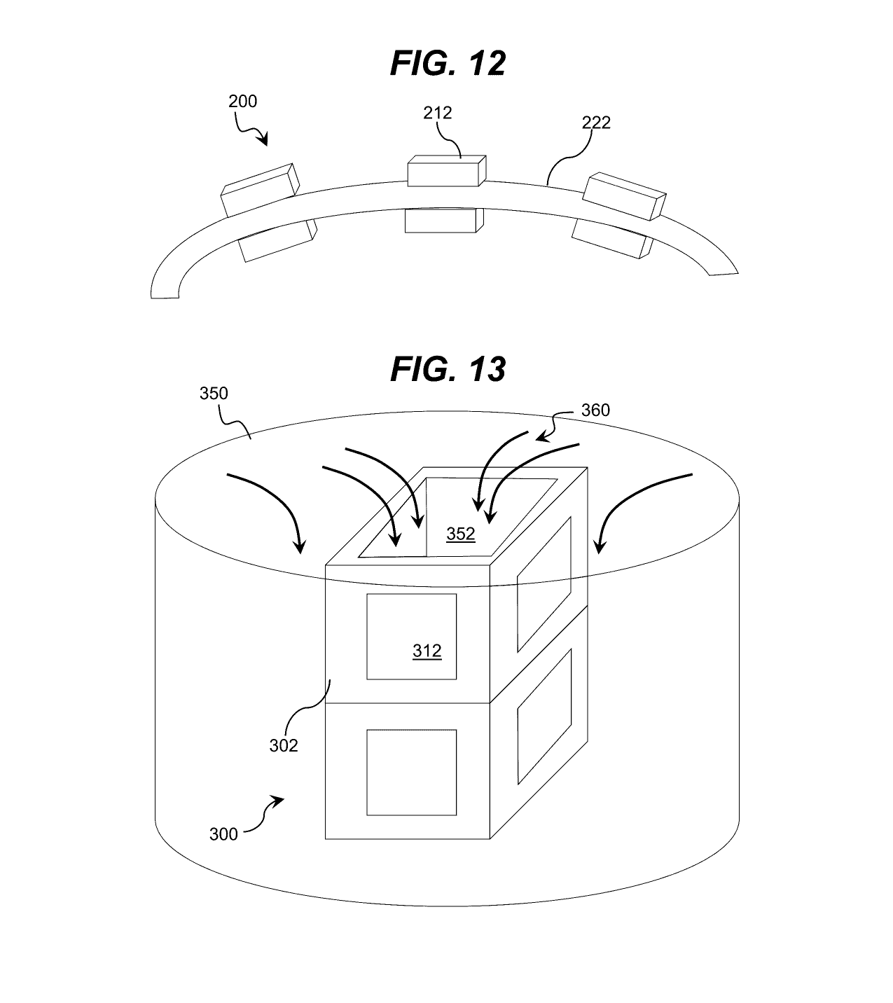

In any of the embodiments provided, it is understood that the contact element or the PCB that connects the set of optoelectronic devices can be flexible. For example, FIG. 12 shows an illustrative optoelectronic device module 200 with a flexible contact element 222 connecting the set of optoelectronic devices 212. In an embodiment, the configuration and details of the optoelectronic device module 200 are similar to the configuration and details of the optoelectronic device module 10 shown in FIG. 1. It is understood that the set of optoelectronic devices 212 each include components that comprise flexible domains for the flexible contact element 222, which can be formed of a flexible material, such as a flexible plastic covered with a metallic film.

The various embodiments of optoelectronic device modules provided herein can be used for a variety of applications, including applications related to the disinfection of liquids. For example, FIG. 13 shows an illustrative optoelectronic device module 300 that is a rectangular cuboid shape. However, it is understood that the module 300 can be any shape. The module 300 is a PCB that includes outer walls 302 with a set of optoelectronic devices 312 mounted on them. In an embodiment, the set of optoelectronic device 312 are UV LEDs that are configured to disinfect a liquid. The module 300 can be placed within a container 350 filled with a liquid. An opening 352 in the module 300 can allow for the liquid to flow 360 through the module 300 in order to improve the heat management of the set of optoelectronic devices 312. The module 300 can eliminate various parts of the liquid due to radiative pattern comprising light radiating largely in all directions.

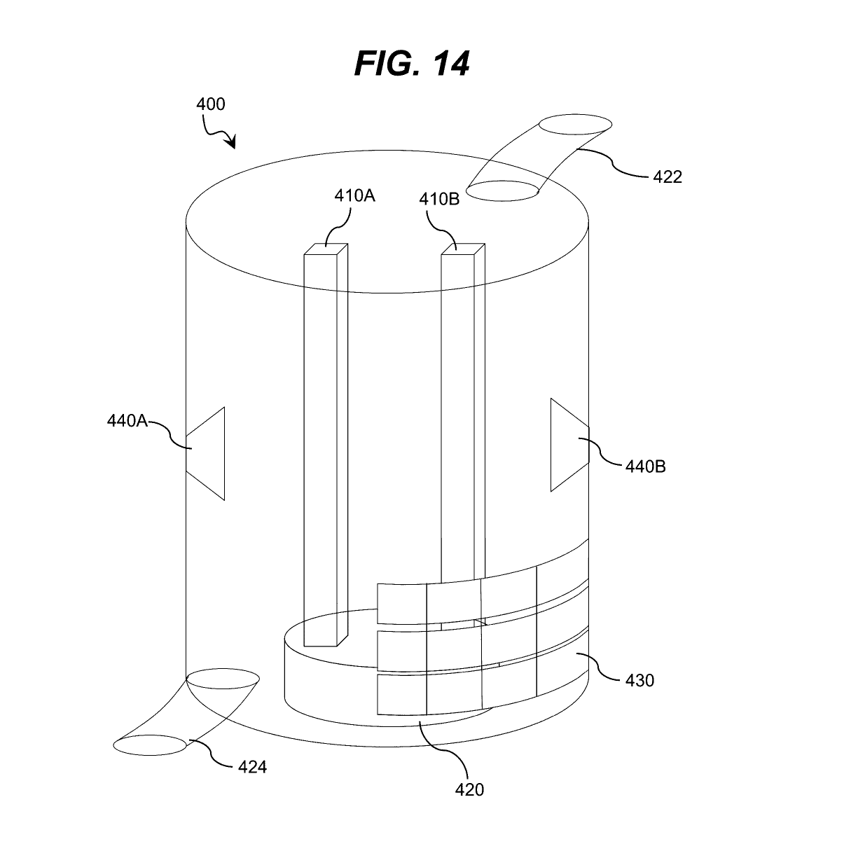

Turning now to FIG. 14, an illustrative disinfection module 400 according to an embodiment is shown. The disinfection module 400 can include a design that incorporates any of the optoelectronic device modules discussed herein as a source of radiation for the disinfection. For example, the module 400 can include a first optoelectronic device module 410A and a second optoelectronic device module 410B, both of which are similar to the module 300 shown in FIG. 13. The device modules 410A, 410B are connected to an electrical module 420 that is configured to deliver and control power for the device modules 410A, 410B. The disinfection module 400 includes an inlet 422 configured to receive a liquid for disinfection and an outlet 424 configured to release the disinfected liquid out of the disinfection module 400.

In an embodiment, the disinfection module 400 can include an array of solar cell elements 430 for providing power to the electrical module 420. The disinfection module 400 can also include a set of sensors 440A, 440B that are configured to determine the transparency of the fluid in order to determine a set of characteristics (wavelength, intensity, time, power, and/or the like) of the radiation provided by the device modules 410A, 410B. Although the disinfection module 400 is only shown with two device modules 410A, 410B, it is understood that any number of device modules can be present. Furthermore, it is understood that other modules can be presented for improved control of the disinfection module 400.

In any of the embodiments discussed herein, the set of optoelectronic devices can include at least one UV LED. In this embodiment, a UV transparent material, such as fluoropolymer, sapphire, fused silica, anodic aluminum oxide (AAO), and/or the like, can encapsulate the optoelectronic device. Also, in any of the embodiments discussed herein, the radiated light can be emitted in all directions, which results in a radiation angle that is approximately 4π steradians. It is understood that particular embodiments of an optoelectronic module results in angular distribution of light intensity that can be non-uniform in all directions. However, the possibility of using several optoelectronic devices within an optoelectronic module, where each have a particular orientation, allows for a wide angular distribution.

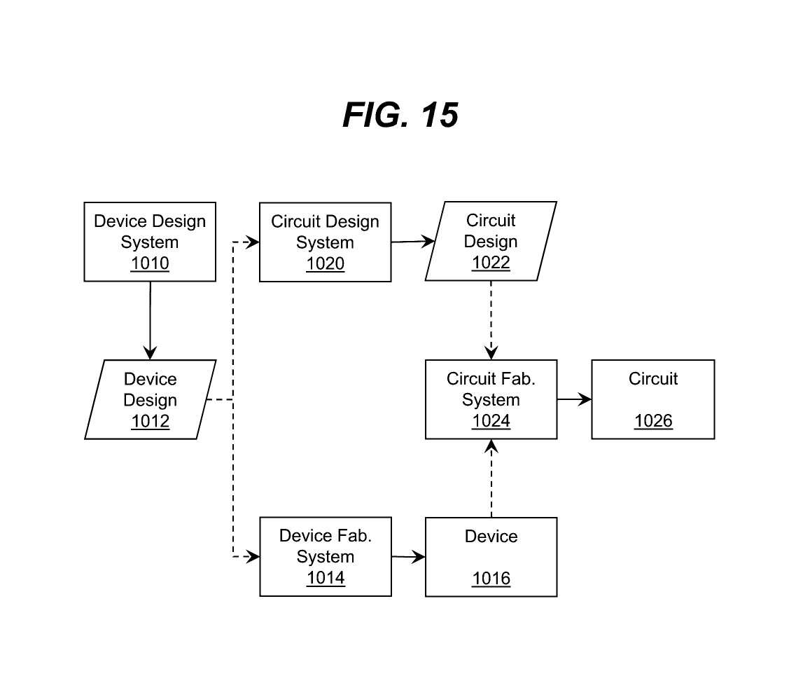

In one embodiment, the invention provides a method of designing and/or fabricating a circuit that includes one or more of the devices designed and fabricated as described herein. To this extent, FIG. 15 shows an illustrative flow diagram for fabricating a circuit 1026 according to an embodiment. Initially, a user can utilize a device design system 1010 to generate a device design 1012 for a semiconductor device as described herein. The device design 1012 can comprise program code, which can be used by a device fabrication system 1014 to generate a set of physical devices 1016 according to the features defined by the device design 1012. Similarly, the device design 1012 can be provided to a circuit design system 1020 (e.g., as an available component for use in circuits), which a user can utilize to generate a circuit design 1022 (e.g., by connecting one or more inputs and outputs to various devices included in a circuit). The circuit design 1022 can comprise program code that includes a device designed as described herein. In any event, the circuit design 1022 and/or one or more physical devices 116 can be provided to a circuit fabrication system 1024, which can generate a physical circuit 1026 according to the circuit design 1022. The physical circuit 1026 can include one or more devices 1016 designed as described herein.

In another embodiment, the invention provides a device design system 1010 for designing and/or a device fabrication system 1014 for fabricating a semiconductor device 1016 as described herein. In this case, the system 1010, 1014 can comprise a general purpose computing device, which is programmed to implement a method of designing and/or fabricating the semiconductor device 1016 as described herein. Similarly, an embodiment of the invention provides a circuit design system 1020 for designing and/or a circuit fabrication system 124 for fabricating a circuit 1026 that includes at least one device 1016 designed and/or fabricated as described herein. In this case, the system 1020, 1204 can comprise a general purpose computing device, which is programmed to implement a method of designing and/or fabricating the circuit 1026 including at least one semiconductor device 1016 as described herein.

In still another embodiment, the invention provides a computer program fixed in at least one computer-readable medium, which when executed, enables a computer system to implement a method of designing and/or fabricating a semiconductor device as described herein. For example, the computer program can enable the device design system 1010 to generate the device design 1012 as described herein. To this extent, the computer-readable medium includes program code, which implements some or all of a process described herein when executed by the computer system. It is understood that the term “computer-readable medium” comprises one or more of any type of tangible medium of expression, now known or later developed, from which a stored copy of the program code can be perceived, reproduced, or otherwise communicated by a computing device.

In another embodiment, the invention provides a method of providing a copy of program code, which implements some or all of a process described herein when executed by a computer system. In this case, a computer system can process a copy of the program code to generate and transmit, for reception at a second, distinct location, a set of data signals that has one or more of its characteristics set and/or changed in such a manner as to encode a copy of the program code in the set of data signals. Similarly, an embodiment of the invention provides a method of acquiring a copy of program code that implements some or all of a process described herein, which includes a computer system receiving the set of data signals described herein, and translating the set of data signals into a copy of the computer program fixed in at least one computer-readable medium. In either case, the set of data signals can be transmitted/received using any type of communications link.

In still another embodiment, the invention provides a method of generating a device design system 110 for designing and/or a device fabrication system 114 for fabricating a semiconductor device as described herein. In this case, a computer system can be obtained (e.g., created, maintained, made available, etc.) and one or more components for performing a process described herein can be obtained (e.g., created, purchased, used, modified, etc.) and deployed to the computer system. To this extent, the deployment can comprise one or more of: (1) installing program code on a computing device; (2) adding one or more computing and/or I/O devices to the computer system; (3) incorporating and/or modifying the computer system to enable it to perform a process described herein; and/or the like.

The foregoing description of various aspects of the invention has been presented for purposes of illustration and description. It is not intended to be exhaustive or to limit the invention to the precise form disclosed, and obviously, many modifications and variations are possible. Such modifications and variations that may be apparent to an individual in the art are included within the scope of the invention as defined by the accompanying claims.

What is claimed is:

1. An optoelectronic device module, comprising:
a first and a second set of optoelectronic devices, wherein each optoelectronic device includes a first contact and a second contact; and
a contact element including a first lateral side and a second lateral side, wherein the first contact of each optoelectronic device in the first set of optoelectronic devices is connected to the first lateral side of the contact element and the first contact of each optoelectronic device in the second set of optoelectronic devices is connected to the second lateral side of the contact element.

2. The optoelectronic device module of claim 1, wherein the first contact is a p-type contact.

3. The optoelectronic device module of claim 1, wherein the first contact is a n-type contact.

4. The optoelectronic device module of claim 1, wherein the contact element is a flexible contact.

5. The optoelectronic device module of claim 1, wherein the contact element is a printed circuit board including a set of openings for each of the optoelectronic devices.

6. The optoelectronic device module of claim 5, wherein each of the optoelectronic devices includes:
a mesa region that forms a p-type electrode that is configured to contact a p-type contact within each of the openings in the set of openings; and
a n-type electrode that surrounds the mesa region and is configured to contact a n-type contact surrounding each of the openings in the set of openings.

7. The optoelectronic device module of claim 6, wherein each of the openings includes a transistor connected to the p-type contact for controlling the on/off characteristics of each optoelectronic device.

8. The optoelectronic device module of claim 7, wherein each optoelectronic device represents a pixel in a thin film transistor (TFT) active matrix network.

9. The optoelectronic device module of claim 1, wherein the first set of optoelectronic devices are located on a first substrate wafer and the second set of optoelectronic devices are located on a second substrate wafer.

10. The optoelectronic device module of claim 1, wherein at least one of the first and second set of optoelectronic devices is an ultraviolet LED.

11. The optoelectronic device module of claim 1, wherein a light emitted by the first and second set of optoelectronic devices is at least 4π steradians.

12. A LED module, comprising:
a first and a second set of LEDs, wherein each LED includes a first contact and a second contact; and
a contact element including a first lateral side and a second lateral side, wherein the first contact of each LED in the first set of LEDs is connected to the first lateral side of the contact element and the first contact of each LEDs in the second set of LEDs is connected to the second lateral side of the contact element, wherein a light emitted by the first and second set of LEDs is at least 4π steradians.

13. The LED module of claim 12, wherein the first contact is a p-type contact.

14. The LED module of claim 12, wherein the first contact is a n-type contact.

15. The LED module of claim 12, wherein the contact element is a flexible contact.

16. The LED module of claim 12, wherein the contact element is a printed circuit board including a set of openings for each of the LEDs, and wherein each of the LEDs includes a mesa region that forms a p-type electrode that is configured to contact a p-type contact within each of the openings in the set of openings, and wherein each of the LEDs includes a n-type electrode that surrounds the mesa region that is configured to contact a n-type contact surrounding each of the openings in the set of openings.

17. The LED module of claim 16, wherein each LED represents a pixel in a thin film transistor (TFT) active matrix network, and wherein each of the openings includes a transistor connected to the p-type contact for controlling the on/off characteristics of each LED.

18. The LED module of claim 12, wherein the first set of LEDs are located on a first substrate wafer and the second set of LEDs are located on a second substrate wafer.

19. The LED module of claim 12, wherein at least one of the first and second set of LEDs is an ultraviolet LED.

20. A disinfection module, comprising:
a container including an inlet for receiving a fluid and an outlet for releasing the fluid contained within the container;
a set of sensors configured to determine a transparency of the fluid within the container; and
a set of optoelectronic device modules for emitting radiation to disinfect the fluid within the container, each of the optoelectronic device modules comprising:
a first and a second set of optoelectronic devices, wherein each optoelectronic device includes a first contact and a second contact; and
a contact element including a first lateral side and a second lateral side, wherein the first contact of each optoelectronic device in the first set of optoelectronic devices is connected to the first lateral side of the contact element and the first contact of each optoelectronic device in the second set of optoelectronic devices is connected to the second lateral side of the contact element.
